(19) United States
(12) Patent Application Publication
(10) Pub. No.: US 2001/0031461 A1
(43) Pub. Date: Oct. 18, 2001

(54) Synthetic lethal expression screen

(76) Inventor: Stuart Tugendreich, Mountain View, CA (US)

Correspondence Address: ROBINS & PASTERNAK LLP, 90 MIDDLEFIELD ROAD, SUITE 200, MENLO PARK, CA, 94025, US

(21) Appl. No.: 09/738,392

(22) Filed: Dec. 15, 2000

Related U.S. Application Data
(60) Provisional application No. 60/171,852, filed on Dec. 22, 1999.

(51) Int. Cl. C12Q 1/00; C12N 15/81

(52) U.S. Cl. 435/4; 435/483

(57) ABSTRACT

A method for potentiating a host cell for sensitivity to a heterologous polynucleotide comprises providing a population of host cells, wherein the cells comprise a heterologous polynucleotide, a first promoter regulating the expression of the heterologous polynucleotide, and a replicable genetic element comprising a second polynucleotide encoding a repressor of the first promoter, a reporter gene under the control of a second promoter, expression of which provides a detectable label, and an origin of replication, wherein the replicable genetic element is subject to loss by the host cells, thus resulting in heterogeneously labeled colonies when the host cells are cultured; mutagenizing the population of host cells; growing colonies of the mutagenized host cells under conditions wherein the heterologous polynucleotide, the repressor, and the reporter are all expressed; and identifying a colony of mutagenized host cells that is homogeneously labeled.

### FIELD OF THE INVENTION

This invention relates generally to the fields of molecular biology and functional genomics. More particularly, the invention relates to methods for constructing and identifying host cells that are sensitive to the activity of a heterologous protein, the cells created and identified thereby, and assays employing the cells.

### BACKGROUND OF THE INVENTION

In surrogate genetics, a host cell is transformed with a heterologous gene, and the host cell is examined for a change in phenotype due to expression of the heterologous gene. If an observable phenotype results, one can then use the resulting transformed cells to assay compounds for the ability to reverse the heterologous protein activity, regardless of whether or not the protein activity is known. However, sometimes expression of the heterologous gene does not provide an observable phenotype. It is sometimes possible to identify a “potentiated” host cell that, due to its genotype, is more susceptible to the heterologous protein activity, but this generally requires a large number of transformations, and is not always successful.

### SUMMARY OF THE INVENTION

I have now invented a method for identifying and creating potentiated host cells, which can immediately be used in further experiments to determine the function of a heterologous gene, and for chemical annotation of the gene.

One aspect of the invention is a method for potentiating a host cell for sensitivity to a heterologous polynucleotide, the method comprising providing a population of host cells, wherein the cells comprise a heterologous polynucleotide, a first inducible promoter regulating the expression of the heterologous polynucleotide, and a separate replicable genetic element comprising a second polynucleotide encoding a repressor of the first promoter, a reporter gene under the control of a second promoter, expression of which provides a detectable label, and an origin of replication, wherein the replicable genetic element is subject to loss by the host cells, thus resulting in heterogeneously labeled colonies when the host cells are cultured; mutagenizing the population of host cells; growing colonies of the mutagenized host cells under conditions wherein the heterologous polynucleotide, the repressor, and the reporter are all expressed; and identifying a colony of mutagenized host cells that is homogeneously labeled.

### DETAILED DESCRIPTION

Definitions:

The term “heterologous polynucleotide” refers to a nucleic acid that is foreign to a selected host cell, or is otherwise altered (for example, a native gene placed under control of a different promoter).

The term “replicable genetic element” refers to a polynucleotide construct such as a plasmid, artificial chromosome, and the like, which can be maintained in a host cell. Replicable genetic elements, as used herein, are subject to spontaneous loss by their host cell unless maintained by selective pressure.

“Heterogeneously labeled” and “sectored” colonies comprise clonally related host cells that express a label encoded on the replicable genetic element, and host cells that have spontaneously discarded the replicable genetic element and fail to express the label.

The term “reporter gene” refers to a polynucleotide that encodes a molecule that can be detected readily, either directly or by its effect on the host cell (phenotype). Exemplary reporter genes encode enzymes, for example the ADE2 or ADE3 gene products, β-galactosidase and URA3, luminescent or fluorescent proteins, such as Green Fluorescent Protein (GFP) and variants thereof, antigenic epitopes (for example Glu-tags), mRNA of distinct sequences, and the like.

The term “selectable marker” refers to a gene that can be used to modulate host cell growth (for example, antibiotic resistance) or facilitate separation of host cells that express the marker from cells that fail to express the marker (for example, GFP, surface antigens, and the like).

The term “fluorescent protein” refers to a protein capable of fluorescing when illuminated. Exemplary fluorescent proteins include, without limitation, the Aequorea victoria “Green Fluorescent Protein” (“GFP”: see for example D. C. Prasher et al., Gene (1992) 111:229-33; M. Chalfie et al., Science (1994) 263:802-05, both incorporated herein by reference), and fluorescent mutants thereof (“GFP variants”: see for example U.S. Pat. No. 5,625,048 and U.S. Pat. No. 5,777,079, both incorporated herein by reference).

General Method:

The method of the invention can be used, in general, with any host cell and any heterologous gene or polynucleotide of interest. In practice, it is presently preferred to employ a host cell that can be grown and cultured easily, and that has other characteristics that make it suitable for use in assays. For example, if one plans to use the product cells in high-throughput assays for drug screening, one can select a host cell strain that lacks proteins capable of ejecting compounds from the cell. The heterologous polynucleotide and its promoter can be provided on a plasmid or artificial chromosome, or can be integrated into the host cell genome. The promoter selected should be functional in the selected host cell, should be inducible (so that later it can be turned “off” so that the repressor plasmid can be removed, and the heterologous gene induced for compound screening), and should be capable of being repressed by a known repressor. Alternatively, the repressor can be located on the inducible promoter, and can be turned off during compound screening. If the heterologous polynucleotide is provided on a plasmid or other vehicle that can be lost by the host, it will preferably further include a selectable marker, so that retention can be assured by culturing on selective media.

A polynucleotide encoding a protein capable of repressing the first promoter is maintained on a second element, such as a plasmid, that can be lost spontaneously by the host cell. This element further includes a promoter that provides for constitutive expression of the repressor (or regulated expression sufficient to suppress the first promoter), and a polynucleotide encoding a detectable marker. The second element is transformed into the host cell, and the cell is cultured under conditions wherein the first promoter would be induced but for the repressor, and the repressor promoter is induced to express sufficient repressor to prevent substantial heterologous protein expression.

After a suitable time period, the cell cultures are examined. If the heterologous protein does not kill or inhibit the host cell growth, then the cells will not require the second genetic element in order to survive, and will spontaneously eliminate it in some cases. This results in cultures (colonies) that are heterogeneously labeled: typically such cultures will appear “sectored”, with labeled sectors containing cells that have retained the labeled repressor plasmid, and non-labeled sectors containing cells (and their descendents) that have lost the label. If all cultures appear homogeneously labeled, the result indicates that the repressor plasmid is required, and suggests that the host cell does not require any further potentiation.

“Sectoring” can be detecting by a variety of different methods, depending in part upon the label selected. For example, where the label is visually observable, one can detect sectoring (heterogeneous labeling) by simple visual or instrumental inspection of host cell colonies. Instrumental inspection can be performed using a photometer or other device capable of measuring light output or absorbance. Other labels may require that one first develop the signal, for example by contacting the host cells with a labeled nucleic acid, a chromogenic substrate, and the like. Alternatively, one can examine heterogeneity by FACS and related methods, for example by suspending individual colonies and separating labeled from non-labeled cells, and comparing the quantity of non-labeled cells with that found in control colonies.

If the typical sectored result is obtained, it indicates that the host cell is not sensitive to the presence of the heterologous protein. The transformed host cells (containing the repressor plasmid) are then mutagenized by any convenient method, cultured again, and reexamined for sectoring. It is expected that some cultures will again present a sectored appearance, while others will appear uniformly labeled. Uniformly labeled colonies result from mutations that render the host cell sensitive to the heterologous protein, thus making them dependent on the repressor plasmid. These cells can be cultured and used as assay cells potentiated for the heterologous polynucleotide used. For example, the host cells can be cultured in the presence of a test compound under conditions that prevent or inhibit expression of the repressor (or after removal of the repressor plasmid): compounds that permit the host cell to grow in the absence of the repressor probably reverse or inhibit the activity of the heterologous protein.

The potentiated cells are also useful indicators of the heterologous gene function. By determining how the potentiated cell differs from the original strain (for example, which enzymes and/or metabolic pathways were affected by mutagenesis), one can identify the enzymes and metabolic pathways that interact with the heterologous gene, and by extrapolation, the homologous enzymes most likely to interact with the heterologous gene in its native environment.

What is claimed:

1. A method for potentiating a host cell for sensitivity to a heterologous polynucleotide, the method comprising:
a) providing a population of host cells, said cells comprising
i) A heterologous polynucleotide;
ii) a first promoter regulating the expression of said heterologous polynucleotide; and
iii) a replicable genetic element comprising
(1) a second polynucleotide encoding a repressor of said first promoter;
(2) a reporter gene under the control of a second promoter, expression of which provides a detectable label; and
(3) an origin of replication, wherein said replicable genetic element is subject to loss by said host cells, thus resulting in heterogeneously labeled colonies when said host cells are cultured;
b) mutagenizing said population of host cells;
c) growing colonies of said mutagenized host cells under conditions wherein said heterologous polynucleotide, said repressor, and said reporter are all expressed; and
d) identifying a colony of mutagenized host cells that is homogeneously labeled.

2. The method of claim 1, wherein said reporter gene comprises ADE2, and said host cells comprise ade⁻yeast.

3. The method of claim 1, wherein said reporter gene encodes GFP.

4. The method of claim 1, wherein said host cells comprise yeast.

5. The method of claim 1, wherein said replicable genetic element comprises a plasmid.

6. The method of claim 1, wherein said heterologous polynucleotide and first promoter are provided on a plasmid.

7. The method of claim 6, wherein said plasmid further comprises a selectable marker.
